(19) United States
(12) Patent Application Publication
(10) Pub. No.: US 2023/0372559 A1
(43) Pub. Date: Nov. 23, 2023

(54) ULTRAVIOLET DOWNLIGHT FOR USE IN DISINFECTING AN ENVIRONMENT FOR HUMAN OCCUPATION

(71) Applicant: CURRENT LIGHTING SOLUTIONS, LLC, East Cleveland, OH (US)

(72) Inventors: Mark E. Kaminski, East Cleveland, OH (US); Steve Germain, Montreal (CA); Beniot Essiambre, Montreal (CA); Yaseen Waheed, Montreal (CA)

(73) Assignee: CURRENT LIGHTING SOLUTIONS, LLC, East Cleveland, OH (US)

(21) Appl. No.: 18/028,268

(22) PCT Filed: Sep. 10, 2021
(86) PCT No.: PCT/US2021/049760

§ 371 Date: Mar. 24, 2023

Related U.S. Application Data
(60) Provisional application No. 63/083,197, filed on Sep. 25, 2020.

(51) Int. Cl. A61L 2/10

(52) U.S. Cl. CPC A61L 2/10; A61L 2202/11; A61L 2202/25

(57) ABSTRACT

A luminaire includes one or more light emitting diodes (LEDs) and a beam-spreading total internal reflection (TIR) optic optically coupled with the one or more LEDs and configured to spread light output by the one or more LEDs. The beam-spreading TIR optic includes a base and an apex and a tapered sidewall extending from a perimeter of the base to the apex, and the one or more LEDs are optically coupled into the base. There may be N LEDs where N is an integer greater than or equal to two, and the beam-spreading TIR optic may further include N optical condensers connected to the base, with each LED optically coupled into the base by a corresponding optical condenser. The luminaire may further include peripheral white LEDs disposed around the beam-spreading TIR optic, which are not optically coupled with the beam-spreading TIR optic. A surrounding annular reflector may further be provided.

This application claims the benefit of U.S. Provisional Application No. 63/083,197 filed Sep. 25, 2020 titled “ULTRAVIOLET DOWNLIGHT FOR USE IN DISINFECTING AN ENVIRONMENT FOR HUMAN OCCUPATION”. U.S. Provisional Application No. 63/083,197 filed Sep. 25, 2020 titled “ULTRAVIOLET DOWNLIGHT FOR USE IN DISINFECTING AN ENVIRONMENT FOR HUMAN OCCUPATION” is incorporated herein by reference in its entirety.

### BACKGROUND

The following relates to the disinfection arts, pathogen control arts, bacterial pathogen control arts, lighting arts, and the like.

Clynne et al., U.S. Pat. No. 9,937,274 B2 issued Apr. 10, 2018 and Clynne et al., U.S. Pat. No. 9,981,052 B2 (which is a continuation of U.S. Pat. No. 9,937,274) provide, in some illustrative examples, disinfection systems that includes a light source configured to generate ultraviolet light toward one or more surfaces or materials to inactivate one or more pathogens on the one or more surfaces or materials.

U.S. Pub. No. 2016/0271281 A1 is the published application corresponding to U.S. Pat. No. 9,937,274. U.S. Pub. No. 2016/0271281 A1 is incorporated herein by reference in its entirety to provide general information on disinfection systems for occupied spaces that use ultraviolet light.

Chinniah et. al., U.S. Pat. No. 9,233,510 titled “Lenses for cosine cubed, typical batwing, flat batwing distributions” discloses a lighting apparatus with uniform illumination distribution, which according to some embodiments includes a lens for area lighting. In one embodiment, the lens comprises a plurality of cross-sections identified by a thickness ratio defined at different angles. The thickness ratio is determined relative to the thickness of the cross-section defined at a center angle of the lens. In another embodiment, the lighting apparatus with uniform illumination distribution includes a lens having an inner surface and an outer surface. A profile of the inner surface and the outer surface is composed of a plurality of piecewise circular arcs defined with radii and circle centers. The lens is formed as a complex curve lens by joining the piecewise circular arcs of the inner surface and the outer surface.

Certain improvements are disclosed.

### BRIEF DESCRIPTION

In some illustrative embodiments disclosed herein, a luminaire includes one or more ultraviolet (UV) light emitting diodes (LEDs) configured to output ultraviolet light, and a beam-spreading total internal reflection (TIR) optic optically coupled with the one or more UV LEDs and configured to spread the ultraviolet light output by the one or more UV LEDs. In some embodiments, the beam-spreading TIR optic includes a base and an apex and a tapered sidewall extending from a perimeter of the base to the apex, and the one or more UV LEDs are optically coupled into the base of the beam-spreading TIR optic. In some embodiments there are N UV LEDs where N is an integer greater than or equal to two. In some embodiments, the beam-spreading TIR optic further includes N optical condensers corresponding to the N UV LEDs. Each optical condenser is connected to the base of the beam-spreading TIR element, and each of the N UV LEDs is optically coupled into the base of the beam-spreading TIR optic by the corresponding optical condenser. In some embodiments, the luminaire may further include peripheral white LEDs configured to emit white light. The peripheral white LEDs are disposed around the beam-spreading TIR optic, and the peripheral white LEDs are not optically coupled with the beam-spreading TIR optic. In some such embodiments, the peripheral white LEDs comprise a ring of peripheral white LEDs, and an annular beam-forming optic is coupled with the ring of peripheral white LEDs. In any of the foregoing embodiments, an annular reflector may surround the beam-spreading TIR optic and the optional peripheral white LEDs.

In some illustrative embodiments disclosed herein, a beam spreading optical element is configured to operate at a design-basis wavelength. The beam spreading optical element includes a tapered TIR optic having a base and an apex and a tapered sidewall extending from a perimeter of the base to the apex, and optical condensers connected to the base of the tapered TIR element. Each optical condenser is configured to condense light of the design-basis wavelength received at an input aperture of the optical condenser into a condensed light beam that passes into the tapered TIR optic and intersects the tapered sidewall of the tapered TIR optic at an angle effective for light beam to be reflected by total internal reflection at the tapered sidewall of the tapered TIR optic. In some embodiments, there are N optical condensers having N corresponding output apertures where N is at least three, and the N optical condensers are connected to the base at a fixed radius from a center of the base and the N optical condensers are circumferentially located around the center of the base at 360°/N intervals. In some such embodiments, the condensed light beams formed by the N optical condensers have mutually parallel optical axes. In some embodiments, the tapered TIR optic has rotational symmetry about a symmetry axis passing through a center of the base and the apex, and the condensed light beam output by each optical condenser is reflected by total internal reflection at the tapered sidewall of the tapered TIR optic into a light distribution having peak intensity at an angle of at least 55 degrees respective to the symmetry axis. The tapered sidewall of the tapered TIR optic optionally has grooves and/or ridges with each groove or ridge extending between the apex and the perimeter of the base.

In some illustrative embodiments disclosed herein, a luminaire includes a beam spreading optical element as set forth in the immediately preceding paragraph, and light emitting diodes (LEDs) coupled with the input apertures of the optical condensers of the beam spreading optical element. In some such luminaires, the LEDs are configured to emit ultraviolet light and the design-basis wavelength is in the range 200-400 nm. In any of the foregoing luminaire embodiments, the luminaire may further include peripheral white LEDs configured to emit white light. The peripheral white LEDs are disposed around the base of the beam spreading optical element, and the peripheral white LEDs are not optically coupled with the beam spreading optical element. Optionally, the peripheral white LEDs form a ring of peripheral white LEDs, and an annular beam forming optic is coupled with the ring of peripheral white LEDs. The annular beam forming optic is separate from the beam spreading optical element. In any of the foregoing embodiments, an annular reflector may surround the beam spreading optical element.

In some illustrative embodiments disclosed herein, a method of manufacturing a beam spreading optical element is disclosed. The beam spreading optical element is molded of a material having a refractive index at a design-basis wavelength. The molding forms the beam spreading optical element as a single molded piece including a tapered TIR optic and N optical condensers connected with the tapered TIR optic where N is at least three. The tapered TIR optic has a base and an apex and a tapered sidewall that extends from a perimeter of the base to the apex, and each optical condenser is connected to the base of the tapered TIR element and is configured to condense light of the design-basis wavelength received at an input aperture of the optical condenser into a condensed light beam that passes into the tapered TIR optic and intersects the tapered sidewall of the tapered TIR optic at an angle effective for light beam to be reflected by total internal reflection at the tapered sidewall of the tapered TIR optic.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 diagrammatically illustrates a side sectional view of a downlight as disclosed herein, along with some design considerations for designing the downlight.

FIG. 2 diagrammatically illustrates an enlarged view of the side sectional view of the downlight of FIG. 1.

FIG. 3 shows a perspective view of the beam-spreading total internal reflection (TIR) optic of the downlight of FIGS. 1 and 2, with the mounting flange omitted to better illustrate the optical condensers.

FIG. 4 diagrammatically illustrates a side sectional view of the downlight of FIGS. 1-3, again omitting the mounting flange, and including ray tracing illustrating operation of the beam-spreading TIR optic.

FIG. 5 plots a batwing light distribution produced by the beam-spreading TIR optic of the downlight of FIGS. 1 and 2.

FIG. 6 plots a three-dimensional representation of the batwing light distribution of FIG. 5.

FIG. 7 shows a perspective view of the beam-spreading TIR optic of the downlight of FIGS. 1 and 2 including the mounting flange.

FIG. 8 shows an end view of the beam-spreading TIR optic of FIG. 7, viewed from the apex side of the beam-spreading TIR optic.

FIG. 9 shows an end view of the beam-spreading TIR optic of FIGS. 7 and 8, viewed from the base side of the beam-spreading TIR optic.

FIG. 10 shows a side-sectional view of the beam-spreading TIR optic of FIGS. 7-9, taken along Section A-A indicated in FIG. 9.

FIG. 11 shows a side-sectional view of the beam-spreading TIR optic of FIGS. 7-9, taken along Section B-B indicated in FIG. 9.

FIG. 12 diagrammatically illustrates a side sectional view of the downlight of FIGS. 1 and 2 including ray tracing illustrating operation of the peripheral white LEDs.

FIG. 13 shows a variant embodiment of the beam-forming optic coupled with the peripheral white LEDs, in which the beam-forming optic is constructed as a single annular beam-forming optic.

FIG. 14 shows a perspective view of a portion of the luminaire, illustrating the use of the annular beam-forming optic of FIG. 13 along with an annular fastener for assembling components of the luminaire.

FIG. 15 diagrammatically illustrates a side sectional view of a downlight for emitting white light only, which employs two rings of white LEDs secured together by an annular fastener similar to that shown in FIG. 14.

### DETAILED DESCRIPTION

All wavelength ranges referred to herein are to be understood as including the endpoint wavelengths.

“Ultraviolet (UV) radiation” or “UV light” pertains to the range between 100 nm and 400 nm, commonly subdivided into UVA, from 320 nm to 400 nm; UVB, from 280 nm to 320 nm; and UVC, from 100 nm to 280 nm. The violet range of light is 380-450 nm. It will be appreciated that as used herein the term “light” is intended to encompass light in the visible light range (typically considered 400-700 nm, or 380-740 nm in some other spectral partitions) and also UV light, as well as near infrared light (up to about 3000 nm).

The Actinic UV hazard exposure limit for exposure to ultraviolet radiation incident upon the unprotected skin or eye applies to exposure within a specified time period, which is typically any 8-hour period. To protect against injury of the eye or skin from ultraviolet radiation exposure produced by a broadband source, the effective integrated spectral irradiance (effective radiant exposure, or effective dose), Eₛ, of the light source shall not exceed 30 J/m². The effective integrated spectral irradiance, Eₛ, is then defined as the quantity obtained by weighting spectrally the dose (radiant exposure) according to the actinic action spectrum value at the corresponding wavelength. One suitable actinic action spectrum is the published IESNA Germicidal action spectrum.

U.S. Pub. No. 2016/0271281 A1 discloses disinfection systems that includes a light source configured to generate ultraviolet light toward one or more surfaces or materials in an environment for human occupancy (e.g. a room in a house or building, sometimes referred to herein for brevity as an “occupied space” although it may or may not actually be occupied at any given time) to inactivate one or more pathogens on the one or more surfaces or materials. As disclosed therein, ultraviolet light within or partly encompassing the UVA range (e.g. 280-380 nm, or in other embodiments 300-380 nm) is particularly effective for inactivating pathogens, especially bacterial pathogens. Without being limited to any particular theory of operation, it is believed that UVA is typically efficacious in inactivating bacteria by depositing its energy in the outer membrane of the cell, or the cell wall, where the energy of the UVA photon is sufficient to create reactive oxygen species (ROS) or to drive other chemical reactions that may cause enough damage to the cell envelope to kill or inactivate the bacterium.

Light at other wavelengths can also be effective for inactivating pathogens of various types and/or in various environments (e.g. bare airborne pathogen, airborne pathogen within breath aerosols, surface-bound pathogens). For example, ultraviolet light in the UVC range can be particularly effective for inactivating viral pathogens. Disinfection systems employing ultraviolet light at multiple wavelengths and/or multiple wavelength ranges is also contemplated, e.g. a combination of UVA and UVC light emitters which can provide inactivation of a range of pathogens of different types (e.g., bacterial, viral, and/or fungal, or various subgroups of these broad pathogen classifications).

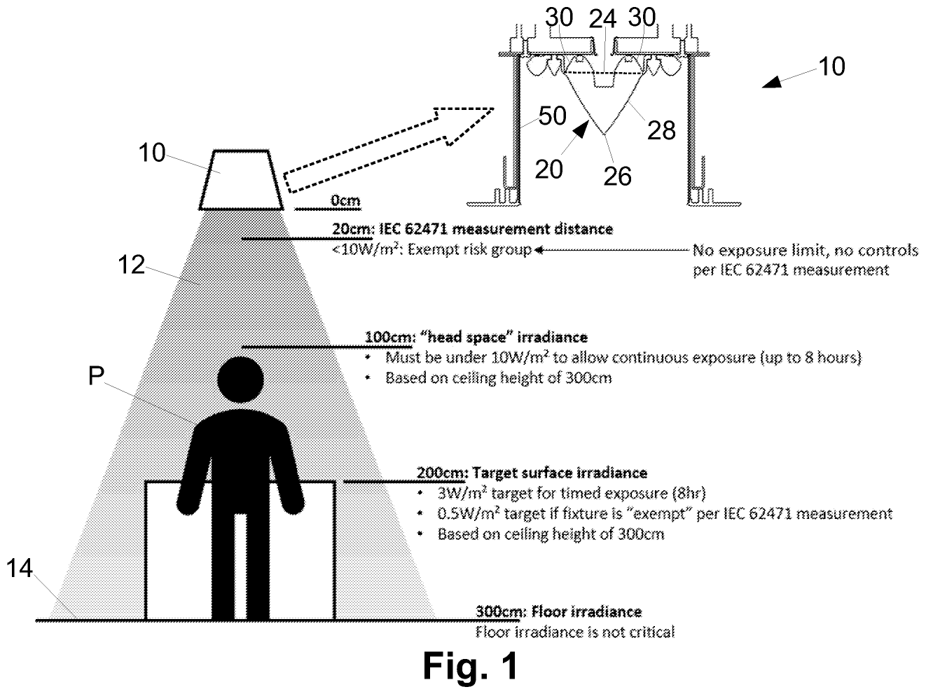

With reference to FIG. 1, a light fixture or luminaire 10 is disclosed. The illustrative luminaire 10 is an illustrative downlight 10 that is ceiling-mounted and emits ultraviolet light 12 from the ceiling into an environment for human occupancy (e.g. an indoor room, closet, hallway, or so forth; or an outdoor space such as a shaded outdoor patio; or an intermediate space such as a semi-enclosed parking garage). In the illustrative environment, a person P of illustrative 200 cm height stands on a floor 14. For the purposes of inactivating pathogens the downlight 10 is desired to provide a design-basis ultraviolet intensity at a target surface that is 100 cm above the floor 14. The downlight 10 is positioned 300 cm above the floor 14, which corresponds to a typical indoor ceiling height.

Measured downward from the downlight 10 (and more precisely from the light output aperture of the downlight 10), therefore, the head level is 100 cm from the downlight 10; the target surface area is 200 cm from the downlight 10; and the floor is 300 cm from the downlight 10. Safety regulations for ultraviolet exposure levels in occupied (or possibly occupied) spaces will typically specify a maximum permissible irradiance (e.g., in watts/meter² or W/m²) at the head level (100 cm from the downlight 10 in the illustrative example) and at the target surface (200 cm from the downlight 10 in the illustrative example). FIG. 1 presents applicable limits for UVA irradiance under the International Electrotechnical Commission (IEC) regulation 62471. Under this IEC 62471 regulation: the “head space” irradiance must be under 10 W/m² to allow for continuous exposure of up to 8 hours; and the target surface irradiance must be 3 W/m² for an 8 hour exposure, assuming a ceiling height of 300 cm. A difficulty with implementing an ultraviolet-emitting downlight to provide for pathogen inactivation under such constraints is that the permissible ultraviolet light irradiance from the downlight depends on the detailed geometry of the space, including factors such as the ceiling height and spacing of the downlights across the ceiling. Hence, the downlight subject to such constraints typically cannot be sold in a retail setting, because the manufacturer has no control over whether the light is installed in a way that meets these constraints. For example, a retail purchaser could install the downlight in a room with a lower 200 cm ceiling, and may thereby fail to meet the above-described IEC 62471 safety regulations. In another example, a retail purchaser could install the luminaire 10 in a fashion other than as a downlight—for example, mounting the luminaire 10 on a wall or floor of a room, thereby potentially placing room occupants into closer proximity to the luminaire 10 and violating the above-described IEC 62471 safety regulations.

Under IEC 62471, there is however an “exempt” class of ultraviolet light sources which do not need to meet these environment-specific constraints. Namely, under IEC 62471, if the downlight 10 emits less than 10 W/m² at a distance of 20 cm from the light output aperture of the downlight 10, then IEC 62471 imposes no other ultraviolet irradiance limits on the downlight 10. Additionally, if this constraint is met that the downlight 10 emits less than 10 W/m² at a distance of 20 cm from the light output aperture of the downlight 10, then there are no electronic exposure controls required for the downlight 10 (e.g., no need to provide control to ensure exposure duration is no longer than 8 hours per day). Hence, if the downlight 10 meets this “exempt” status by emitting less than 10 W/m² at a distance of 20 cm from the light output aperture of the downlight 10, then the downlight 10 can, for example, be sold in a retail setting, and the retail purchaser could mount the downlight 10 on a common ceiling height of 8 feet (or some other lower ceiling height).

In summary, the measured irradiance at 20 cm from the downlight 10 (or, more generally, luminaire 10) needs to be less than 10 W/m² in order to classify as Exempt under IEC 62471. Since downlights are typically small in diameter (for example, 15 cm in diameter) this greatly restricts how much UVA light can be emitted, especially if the UVA light is formed into a beam as is typically the case with a conventional downlight design. A conventional narrow-beam or even a wide-beam downlight creates a hotspot at 20 cm that limits total irradiated watts per fixture to a range of 0.2 to 1.2 Watts. This low wattage ultraviolet output, in turn, greatly reduces the ultraviolet irradiance at the target surface, to a value that is often too low for effective pathogen inactivation.

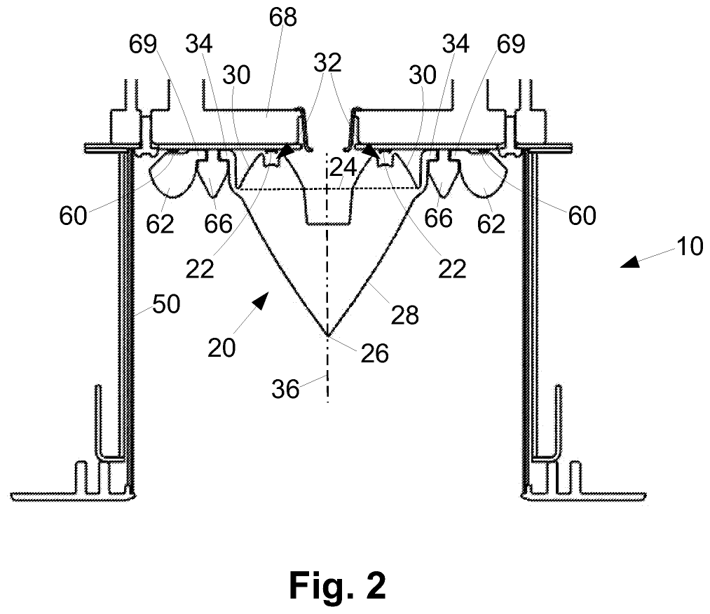

With continuing reference to FIG. 1 and with further reference to FIGS. 2-11 to overcome these difficulties the luminaire 10 (which, again, is shown in an illustrative downlight configuration) includes a beam-spreading total internal reflection (TIR) optic 20 that is optically coupled with one or more UV LEDs 22 (labeled only in FIG. 2) that are configured to output ultraviolet light. The beam-spreading TIR optic 20 is an optical element that is configured to spread the ultraviolet light output by the one or more UV LEDs 22 by reflecting the ultraviolet light via total internal reflection into a batwing light distribution. The beam-spreading TIR optic 20 includes a tapered TIR optic having a base 24 and an apex 26 and a tapered sidewall 28 extending from a perimeter of the base 24 to the apex 26. The beam-spreading TIR optic 20 further includes optical condensers 30 corresponding to the UV LEDs 22. Each optical condenser 30 is connected to the base 24 of the beam-spreading TIR element 20, and each UV LED is optically coupled into the base 24 of the beam-spreading TIR optic 20 by its corresponding optical condenser 30. More particularly, each optical condenser 30 is configured to condense light of a design-basis wavelength (e.g., an ultraviolet wavelength in a range of 200-400 nm in the case of the LEDs 22 being UV LEDs 22 that emit ultraviolet light) received at an input aperture 32 of the optical condenser 30 into a condensed light beam that passes into the tapered TIR optic portion of the beam-spreading TIR optic 20 and intersects the tapered sidewall 28 at an angle effective for light beam to be reflected by total internal reflection at the tapered sidewall 28.

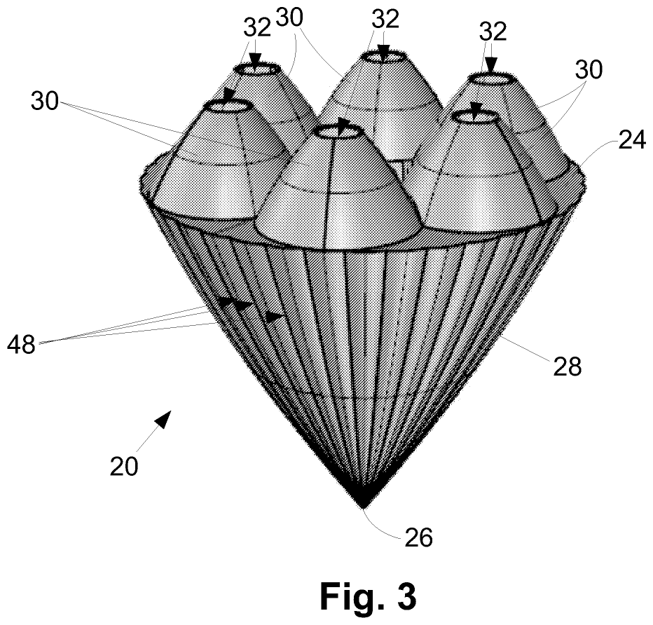

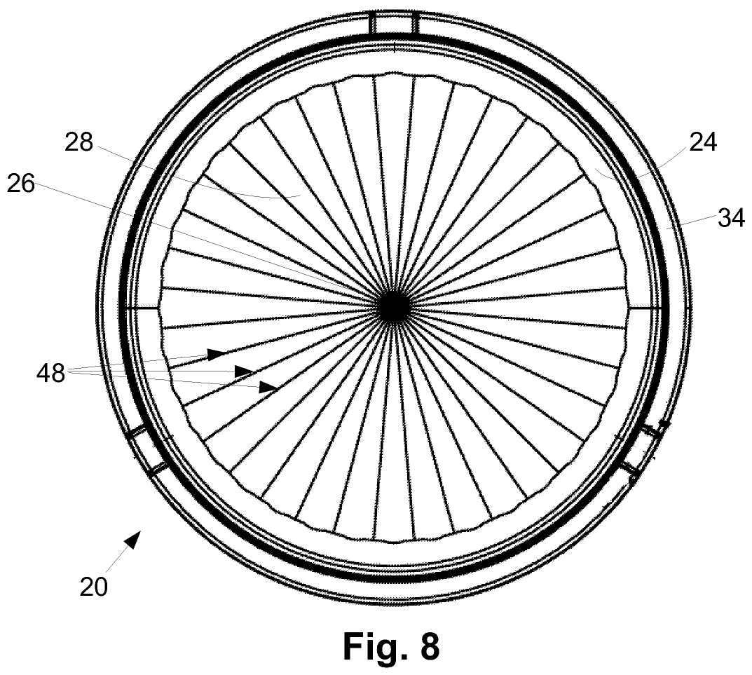

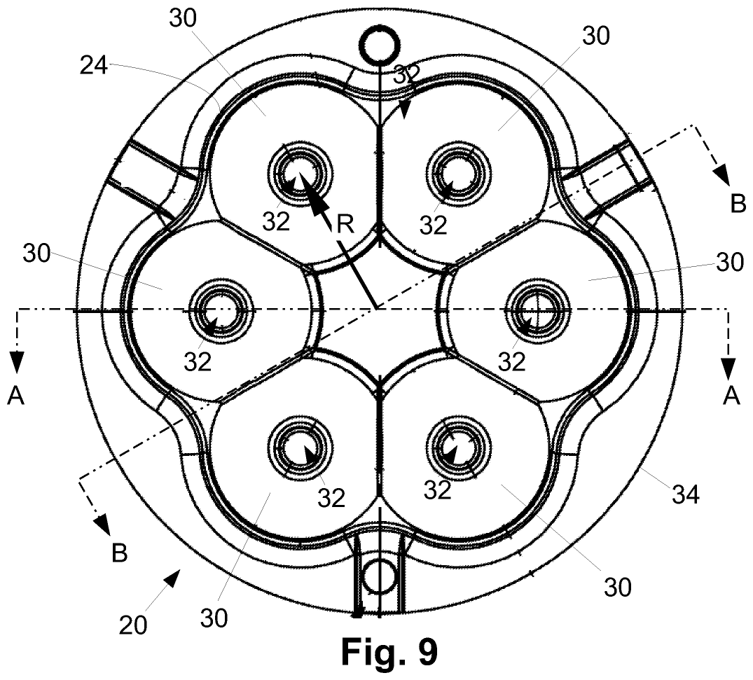

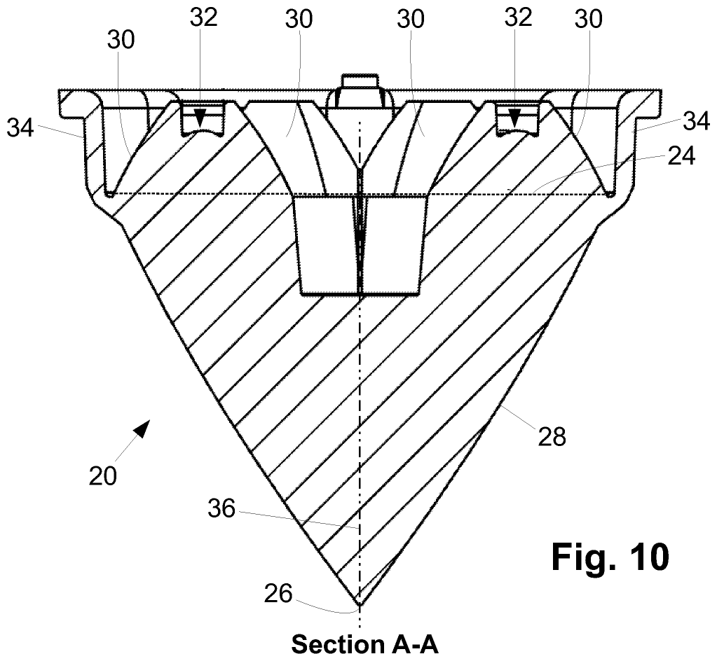

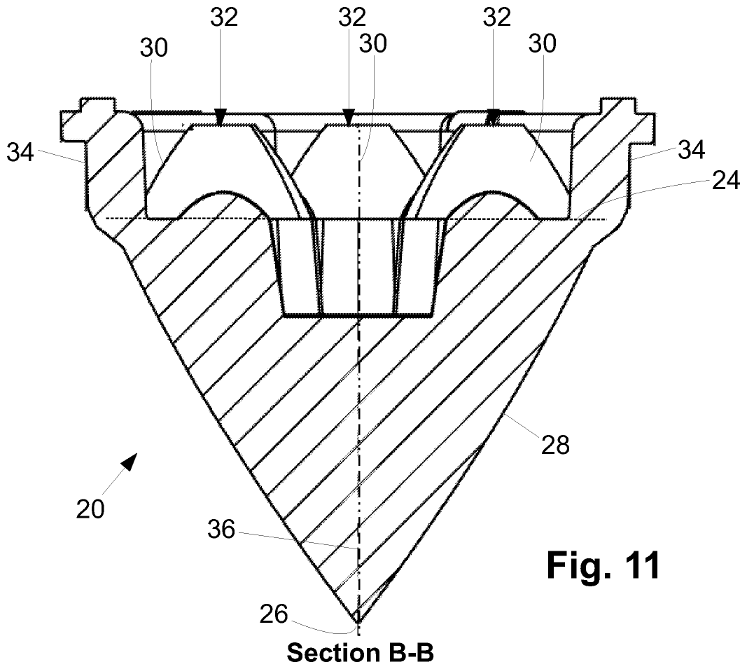

The beam-spreading TIR optic 20 optionally further includes a mounting flange 34, as labeled in FIGS. 2 and 7-11. Note that FIG. 3 omits the mounting flange to better illustrate the optical condensers 30. FIGS. 7-11 provide various views of the beam-spreading TIR optic 20 including the flange 34. FIG. 7 shows a perspective view of the beam-spreading TIR optic 20. FIG. 8 shows an end view of the beam-spreading TIR optic 20, viewed from the apex side (that is, looking at the apex 26). FIG. 9 shows another end view of the beam-spreading TIR optic 20, this time viewed from the base side (that is, looking at the base 24). FIG. 10 shows a side-sectional view of the beam-spreading TIR optic 20 taken along Section A-A indicated in FIG. 9. FIG. 11 shows a side-sectional view of the beam-spreading TIR optic 20 taken along Section B-B indicated in FIG. 9.

As best seen in FIG. 3 which omits the mounting flange, and in the end view from the base side shown in FIG. 9, the illustrative beam-spreading TIR optic 20 includes six optical condensers 30 for coupling a corresponding six UV LEDs 22 into the base 24 of the beam-spreading TIR element 20. More generally, the beam-spreading TIR optic 20 can include N optical condensers 30 corresponding to the N UV LEDs, with each optical condenser 30 being connected to the base 24 of the beam-spreading TIR element 20 and each of the N UV LEDs 22 being optically coupled into the base 24 of the beam-spreading TIR optic 20 by its corresponding optical condenser 30, and more specifically by the UV LED 22 being coupled into the input aperture 32 of its optical condenser 30. It is contemplated for the number N of optical condensers 30 to be as small as N=1; however, more typically, N is equal to or greater than two, and in some preferred embodiments N is equal to or greater than three so as to provide light more distributed in circumferentially around the beam-spreading TIR element.

The tapered TIR optic of the illustrative beam-spreading TIR element 20 has rotational symmetry about a symmetry axis 36 that passes through a center of the base 24 and the apex 26. The symmetry axis 36 is labeled in FIGS. 2, 4, 10, and 11. As best seen in FIGS. 3 and 9, the N optical condensers 30 are connected to the base 24 at a fixed radius R from a center of the base (radius R is indicated only in FIG. 9), and the N optical condensers 30 are circumferentially located around the center of the base at 360°/N intervals. For N optical condensers 30, the rotational symmetry about the symmetry axis 36 of the beam-spreading TIR element 20 as a whole may be N-fold rotational symmetry. The tapered TIR optic of the beam-spreading TIR element 20 (that is, without considering the optical condensers 30) may have continuous rotational symmetry about the symmetry axis 36, as in the illustrative embodiments, or may also have N-fold rotational symmetry (e.g., in this variant with N=6, the tapered sidewall 28 would form a six-sided pyramid, potentially with the six facets of the pyramid being non-planar).

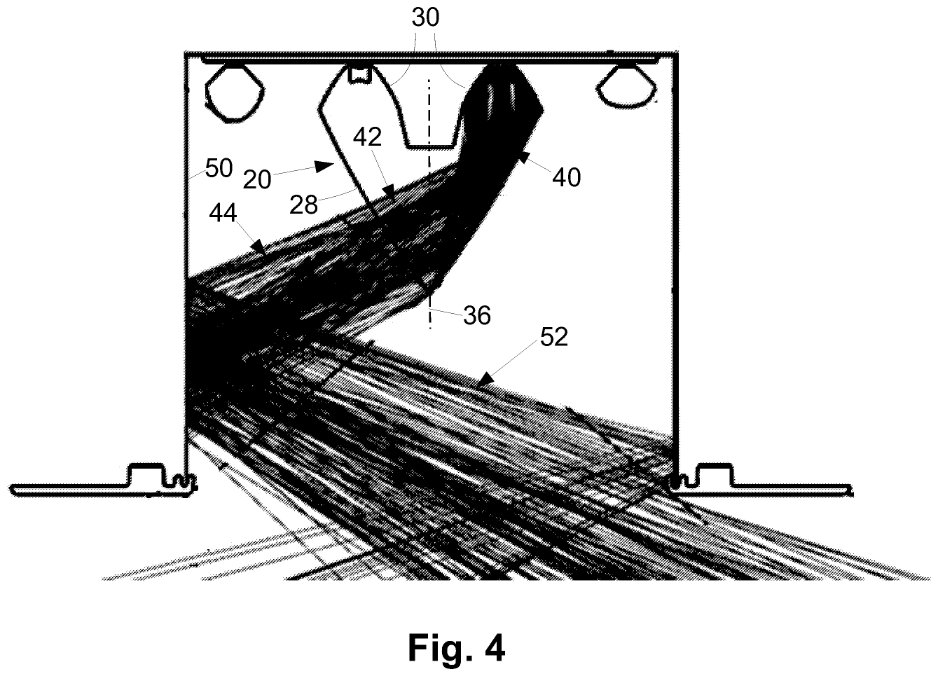

With particular reference to FIGS. 4, 5, and 6, optical operation of the beam-spreading TIR element 20 is illustrated. FIG. 4 shows the basic operation by way of an optical ray tracing diagram following light produced by one of the six UV LEDs 22. Light of the design-basis wavelength (e.g. an ultraviolet wavelength in the case of UV LEDs 22) produced by the illustrative UV LED 22 is condensed by its optical condenser 30 into a condensed light beam 40 that passes into the tapered TIR optic formed by the tapered sidewall 28 extending from the perimeter of the base 24 to the apex 26. The condensed light beam 40 is parallel with, or close to parallel with, the symmetry axis 36. Additionally, if there are multiple (e.g. N) UV LEDs 22 with corresponding optical condensers 30, then the condensed light beams 40 formed by the N optical condensers 30 may optionally have mutually parallel optical axes. It is to be appreciated that while the light beam 40 is condensed, it is not necessarily (and generally is not) perfectly collimated. This condensed light 40 intersects the tapered sidewall 28 at an angle effective for light beam 40 to be reflected by total internal reflection at the tapered sidewall 28 into an angle that is much larger respective to the symmetry axis 36. For example, in some embodiments the condensed light 40 intersects the tapered sidewall 28 at an angle effective for light beam 40 to be reflected by total internal reflection at the tapered sidewall 28 into light 42 with a light distribution having peak intensity at an angle of at least 55 degrees respective to the symmetry axis. In some embodiments, the condensed light 40 intersects the tapered sidewall 28 at an angle effective for light beam 40 to be reflected by total internal reflection at the tapered sidewall 28 into the light 42 with a light distribution having peak intensity at an angle of between 55 degrees and 75 degrees, again measured respective to the symmetry axis 36. It will be noted that due to the geometry of the tapered sidewall 28, this light 42 impinges on the opposite side of the tapered sidewall 28 at a near-normal incidence, and thus exits the beam-spreading TIR element 20 with the light distribution not being significantly modified (although some small angle change due to refraction at the air interface may be designed), as light 44.

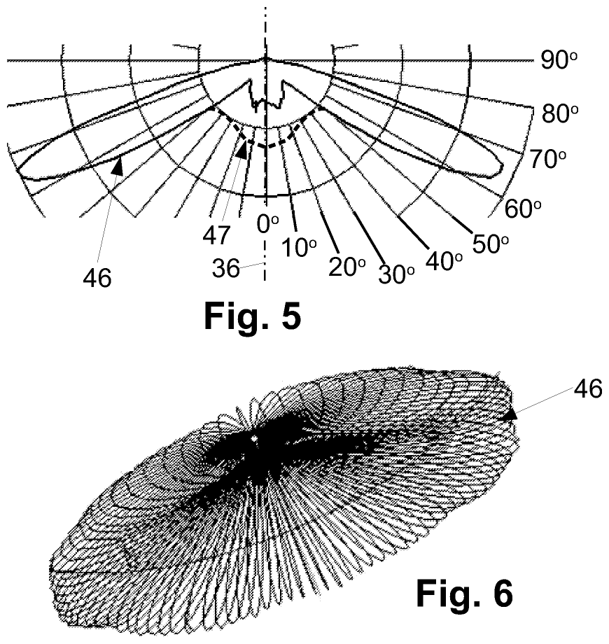

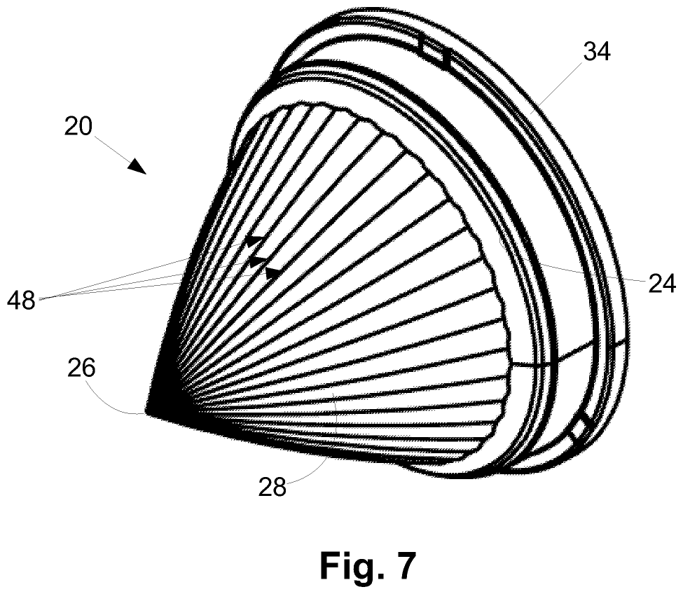

While FIG. 4 shows the ray tracing for a single LED 22 feeding into a single optical condenser 30, this process simultaneously occurs at each of the N optical condensers 30, resulting in a batwing light distribution for N of at least three, and preferably N=4, 5, or 6 or more. The resulting batwing light distribution produced by the total internal reflection at the tapered sidewall 28 preferably has peak intensity at an angle of at least 55 degrees respective to the symmetry axis, and in some embodiments has peak intensity at an angle of between 55 degrees and 75 degrees, again measured respective to the symmetry axis 36. For the illustrative example of FIG. 5 in which N=6, the illustrative batwing light distribution 46 generated by an optical ray tracing simulation of the beam-spreading TIR optic 20 has a batwing light distribution with peak angle at 65 degrees. In contrast to the simulation, some actually constructed embodiments exhibit a higher light intensity contribution at small angles close to the nadir (0 degrees), as diagrammatically shown by the dashed light distribution component 47, although the distribution is still dominated by the large-angle batwing lobe light distribution components. FIG. 6 plots a three-dimensional representation of the batwing light distribution 46 of FIG. 5. Note that in FIG. 5, the line at angle 0° corresponds to the symmetry axis 36, and the angle labels (0°, 10°, 20°, 30°, 40°, 50°, 60°, 70°, 80°, 90°) are respective to the symmetry axis 36.

As best seen in FIGS. 3, 7, and 8, the tapered sidewall 28 optionally has grooves and/or ridges 48, with each groove or ridge 48 extending between the apex 26 and the perimeter of the base 24. The grooves and/or ridges 48 provide radial averaging to smooth out the batwing light distribution 46.

The beam-spreading TIR optic 20 can be manufactured of silicone, acrylic or polymethyl methacrylate (PMMA), glass, or another material that is transparent (or at least translucent) at the design-basis wavelength (e.g., an ultraviolet wavelength in the case of operation with the UV LEDs 22). In one embodiment, the beam spreading TIR optic 20 is formed as a single element in which the tapered TIR optic defined by the tapered sidewall 28 extending between the apex 26 and the perimeter of the base 24 and the N optical condensers 30 are integrally formed together, for example by being molded as a single element. The angle of the tapered sidewall 28 can be selected to ensure the requisite total internal reflection of the condensed light beam 40 (see FIG. 4 and related discussion herein). This can be done using Snell's law of refraction where:

n_{amb}·sin(θ_{amb})=n_{optic}·sin(θ_{optic}) (1)

where n_{amb} and n_{optic} are the refractive indices of the ambient (n_{amb}=1 for air) and the material of the beam-spreading TIR optic 20, respectively, and θ_{amb} and θ_{optic} are the angle of light relative to the surface normal in the ambient and in the beam-spreading TIR optic 20, respectively. Then the minimum angle, θ_{min,TIR}, measured off the surface normal for total internal reflection is given by:

$${θ}_{min , TIR} = arc sin ( \frac{{n}_{a m b}}{{n}_{optic}} ) = arc sin ( \frac{1}{{n}_{o p t i c}} ) \qquad ( 2 )$$

where the rightmost expression assumes n_{amb}=1. So, for example, if the material comprising the beam-spreading TIR optic 20 is silicone having a refractive index of n_{optic}=1.46 then θ_{min,TIR}=43 degrees. Referring back to FIG. 4, this is the minimum angle at which the light 40 impinges on the tapered sidewall 28 to produce total internal reflection. This assumes that the peak intensity output by the optical condenser 30 is parallel to the symmetry axis 36. Since the light 40 output by the optical condenser 30 is typically not perfectly collimated and hence has rays spanning a small distribution of angles centered around the direction parallel to the symmetry axis 36, the angle effective for the light beam 40 to be mostly or entirely reflected by total internal reflection at the tapered sidewall 28 should be somewhat larger than the value of θ_{min,TIR} calculated by Equation (2).

The detailed shape of the optical condensers 30 and the tapered portion of the beam-spreading TIR optic 20 defined by the tapered sidewall 28 can be adjusted based on ray tracing simulations to provide a desired batwing light distribution for a given design basis wavelength and refractive index at that design-basis wavelength of the material making up the beam-spreading TIR optic 20. For example, the illustrative tapered sidewall 28 is not linear but has some curvature as it extends between the perimeter of the base 24 and the apex 26, as best seen in the side sectional views of FIGS. 10 and 11. The apex 26 of the illustrative embodiment comes to a precise point—however, the apex can be rounded or even flat if such geometry simplifies manufacturing. In this regard, however, a large-area flat apex may produce an unwanted hot spot near 0° in the batwing light distribution due to light from the LEDs 22 passing directly through the flat apex. Any such optical “deviations” at the apex 26 can optionally be prevented by adding an opaque coating over the apex 26.

With reference back to FIGS. 1, 2, and 4, the luminaire 10 optionally further includes an annular reflector 50 surrounding the beam-spreading TIR optic 20 and arranged to reflect at least a portion of the ultraviolet light spread by the beam-spreading TIR optic 20. This reflection is best seen in FIG. 4 where the light 44 is reflected by the annular reflector 50 to form reflected light 52. The optional annular reflector 50 may be merely cosmetic, or may provide a mechanism for shaping the edge of the spread beam of ultraviolet light produced by the luminaire 10. While the illustrative annular reflector 50 has straight sidewalls, it is contemplated for the annular reflector to have sidewalls that are slanted (either inward or outward) and/or have some curvature.

The illustrative example of the UV LEDs 22 and the beam-spreading TIR optic 20 is designed for ultraviolet light where the design-basis wavelength is in the wavelength range 200-400 nm, and in some embodiments more specifically for UVA light where the design-basis wavelength is in the wavelength range 280-380 nm, or in other embodiments 300-380 nm. The resulting luminaire 10 thus outputs ultraviolet light for use in inactivating one or more target pathogens (e.g., bacteria or sub-groups of bacteria, viruses or sub-groups of viruses, pathogenic molds or sub-groups of pathogenic molds, various combinations thereof, and/or so forth).

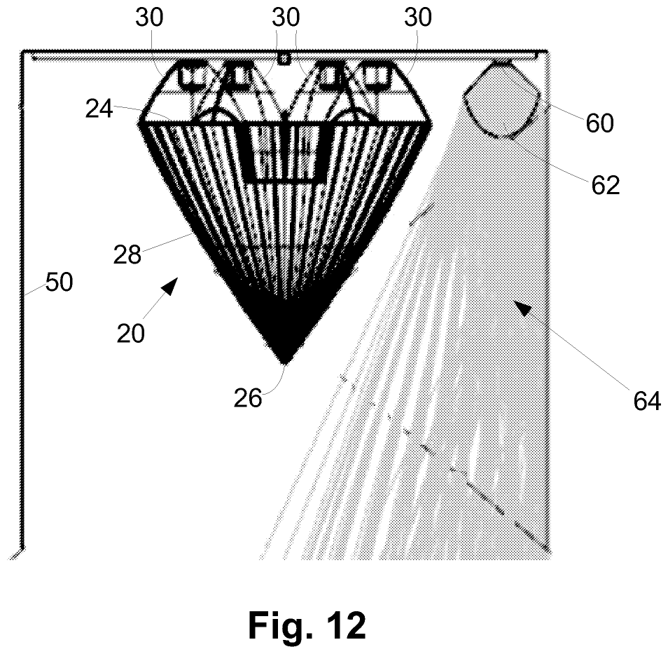

With reference back to FIG. 2 and with further reference to FIG. 12, In some embodiments, it may be desirable for the luminaire 10 to additionally output white light to provide room illumination. This can be advantageous as it allows for a single luminaire 10 to provide both ultraviolet disinfection and room lighting. In the embodiment of FIG. 2 this is provided by peripheral white LEDs 60 which are configured to emit white light (e.g., comprising gallium nitride LEDs coated by a white phosphor in one example embodiment). The peripheral white LEDs 60 are disposed around the beam-spreading TIR optic 20. Preferably, if there are M peripheral white LEDs 60 (where M is at least one, and is more preferably three or more) then they are disposed at regular angular intervals of 360°/M around the beam-spreading TIR optic 20, although some deviation from this regularity of angular intervals is contemplated to, for example, avoid interfering with fasteners of the luminaire 10. The peripheral white LEDs 60 are not optically coupled with the beam-spreading TIR optic 20. Rather, in some embodiments, a separate beam-forming optic 62 is optically coupled with each peripheral white LED 60. As shown by ray tracing in FIG. 12, the coupled beam-forming optic 62 is configured to form the white light emitted by the coupled white LED 60 into a beam 64.

As further seen in FIG. 12, the peripheral location of the peripheral white LEDs 60 places them close to the edge of the annular reflector 50 which surrounds the peripheral white LEDs 60. In some embodiments, the annular reflector 50 reflects at least a portion of the white light to define an edge of the composite white light beam. In other embodiments, the annular reflector 50 (if included at all) does not appreciably contribute to the white light beam shape. Although FIG. 12 shows ray tracing of the beam 64 for only a single peripheral white LED 60 and coupled beam-forming optic 62, it will be appreciated that if there are M peripheral white LEDs 60 and respective coupled beam-forming optics 62, then the resulting white light distribution will be annular; and if M is reasonably large (e.g., M=6 in some embodiments), then the reflections of the beams 64 off the annular reflector 50 will cause the M beams 64 to cross, thereby producing a white light beam that is narrow-beam or relatively narrow-beam.

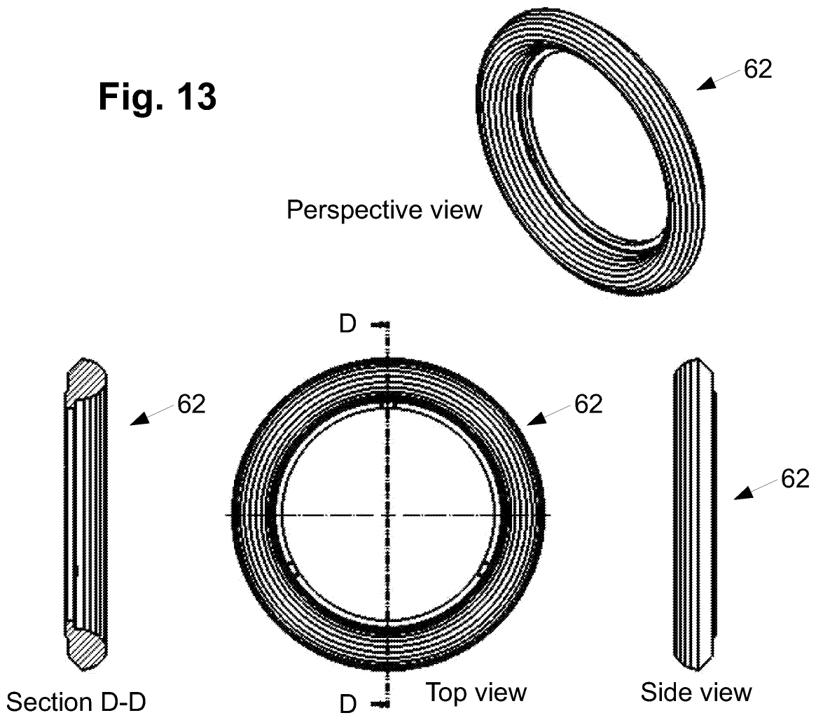

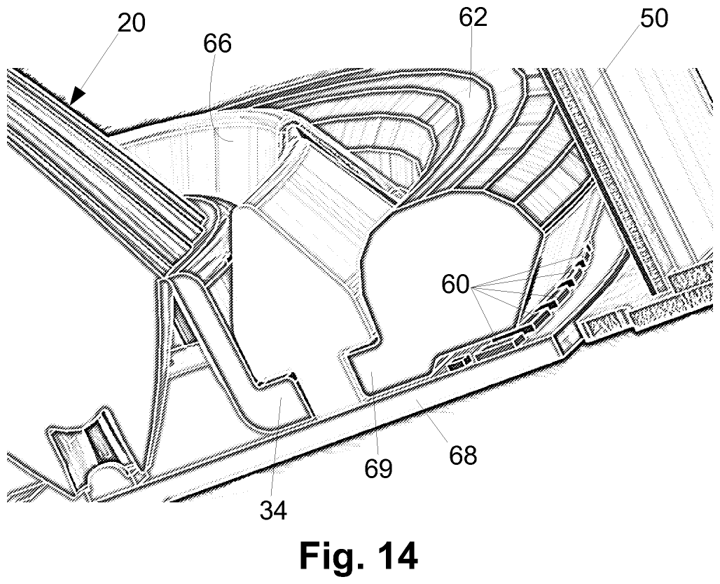

With reference to FIGS. 13 and 14, in another embodiment, the beam-forming optic 62 is constructed as a single annular refractive element that is coupled with a corresponding ring of the peripheral white LEDs 60. FIG. 13 shows perspective, top, and side views of the annular beam-forming optic, along with a sectional view along a Section D-D indicated in the top view. FIG. 14 illustrates a partial perspective view of the annular beam-forming optic 62 coupled with the ring of peripheral white LEDs 60, in the context of the beam-spreading TIR optic 20 and the annular reflector 50.

With reference back to FIG. 2 and with further reference to FIG. 14, in one embodiment one or more fasteners 66 secure both the beam-spreading TIR optic 20 and the annular beam-forming optic 62 to an underlying planar substrate 68, which may for example comprise a dielectric support board or a printed circuit board. The one or more fasteners 66 could be a set of (for example, conical) fasteners distributed around the outside of the perimeter of the base 24 of the beam-spreading TIR optic 20. Alternatively, as seen in FIG. 14, the one or more fasteners 66 could be a single annular fastener 66 that encircles the outside of the perimeter of the base 24 of the beam-spreading TIR optic 20 and in turn is encircled by the annular beam-forming optic 62. The annular fastener 66 captures the mounting flange 34 of the beam-spreading TIR optic 20 and also captures a similar mounting flange 69 of the annular beam-forming optic 62, thereby securing both components 20, 62 with the single annular fastener 66. It will be appreciated that this is merely one illustrative approach for securing the various components together to form the luminaire 10, and other fastening/securing arrangements are also contemplated.

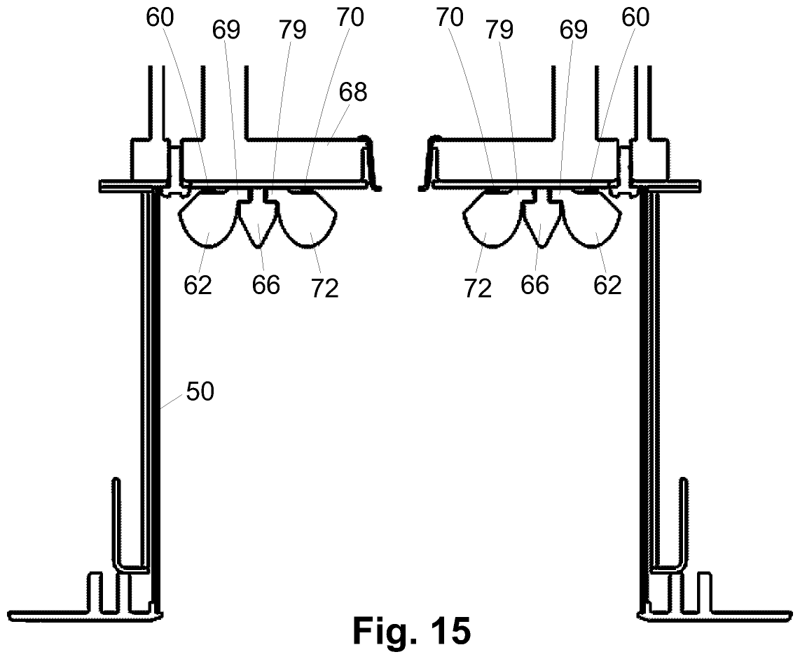

With reference to FIG. 15, if it is desired to provide a luminaire that produces only white light (no disinfection ultraviolet light in this variant embodiment), then two or more rings of white LEDs can be constructed similarly to the arrangement of FIG. 14. The white luminaire of FIG. 15 includes the ring of peripheral white LEDs 60 coupled with the annular beam-forming optic 62 of FIGS. 13 and 14, with the mounting flange 69 of the annular beam-forming optic 62 secured to the support 68 by the annular fastener 66 as shown in FIG. 14. Additionally, a second, inner ring of white LEDs 70 is positioned inboard of the ring of peripheral white LEDs 60. The inner ring of white LEDs 70 is coupled with an inner annular beam-forming optic 72. The inner annular beam-forming optic 72 also has a mounting flange 79 that is secured to the support 68 by the annular fastener 66. It will be appreciated that further inboard rings of LEDs with coupled annular beam-forming optics can be similarly added, as appropriate to provide a desired intensity of white light. Optionally, the annular reflector 50 may surround the rings of white LEDs 60, 70 and coupled annular beam-forming optics 62, 72. Again, the optional annular reflector 50, if included, may be merely cosmetic or may provide an optical function of shaping the edge of the beam of white light produced by the white luminaire of FIG. 15.

The present disclosure has been described with reference to exemplary embodiments. Modifications and alterations may occur to others upon reading and understanding the preceding detailed description. It is intended that the present disclosure be construed as including all such modifications and alterations insofar as they come within the scope of the appended claims or the equivalents thereof.

1. A luminaire comprising:
one or more ultraviolet (UV) light emitting diodes (LEDs) configured to output ultraviolet light; and
a beam-spreading total internal reflection (TIR) optic optically coupled with the one or more UV LEDs and configured to spread the ultraviolet light output by the one or more UV LEDs.

2. The luminaire of claim 1 wherein:
the beam-spreading TIR optic includes a base and an apex and a tapered sidewall extending from a perimeter of the base to the apex, and
the one or more UV LEDs include N UV LEDs that are optically coupled into the base of the beam-spreading TIR optic, where N is an integer greater than or equal to two; and
the beam-spreading TIR optic further includes N optical condensers corresponding to the N UV LEDs, each optical condenser being connected to the base of the beam-spreading TIR element and each of the N UV LEDs being optically coupled into the base of the beam-spreading TIR optic by the corresponding optical condenser.

3-4. (canceled)

5. The luminaire of claim 2 wherein the N optical condensers are connected to the base at a fixed radius from a center of the base and the N optical condensers are circumferentially located around the center of the base at 360°/N intervals.

6. The luminaire of claim 5 wherein the beam spreading TIR optic is formed as a single element.

7. The luminaire of claim 1 wherein:
the beam-spreading TIR optic includes a base and an apex and a tapered sidewall extending from a perimeter of the base to the apex, and the one or more UV LEDs are optically coupled into the base of the beam-spreading TIR optic; and
the tapered sidewall of the beam-spreading TIR optic has grooves and/or ridges with each groove or ridge extending between the apex and the perimeter of the base.

8. The luminaire of claim 1 further comprising:
peripheral white LEDs configured to emit white light, the peripheral white LEDs being disposed around the beam-spreading TIR optic, wherein the peripheral white LEDs are not optically coupled with the beam-spreading TIR optic.

9. The luminaire of claim 8 wherein the peripheral white LEDs form a ring of peripheral white LEDs, the luminaire further comprising an annular beam-forming optic coupled with the ring of peripheral white LEDs.

10. The luminaire of claim 8 further comprising:
an annular reflector surrounding the beam-spreading TIR optic and the peripheral white LEDs.

11. The luminaire of claim 1 wherein:
the tapered TIR optic has rotational symmetry about a symmetry axis passing through a center of the base and the apex; and
the beam-spreading TIR optic reflects the ultraviolet light output by the one or more UV LEDs into a light distribution having peak intensity at an angle of at least 55 degrees respective to the symmetry axis.

12. (canceled)

13. A beam spreading optical element configured to operate at a design-basis wavelength, the beam spreading optical element comprising:
a tapered total internal reflection (TIR) optic having a base and an apex and a tapered sidewall extending from a perimeter of the base to the apex; and
optical condensers connected to the base of the tapered TIR element, each optical condenser configured to condense light of the design-basis wavelength received at an input aperture of the optical condenser into a condensed light beam that passes into the tapered TIR optic and intersects the tapered sidewall of the tapered TIR optic at an angle effective for light beam to be reflected by total internal reflection at the tapered sidewall of the tapered TIR optic.

14. The beam spreading optical element of claim 13 wherein:
there are N optical condensers having N corresponding output apertures where N is at least three; and
the N optical condensers are connected to the base at a fixed radius from a center of the base and the N optical condensers are circumferentially located around the center of the base at 360°/N intervals.

15. The beam spreading optical element of claim 14 wherein the condensed light beams formed by the N optical condensers have mutually parallel optical axes.

16. The beam spreading optical element of claim 13 wherein:
the tapered TIR optic has rotational symmetry about a symmetry axis passing through a center of the base and the apex; and
the condensed light beam output by each optical condenser is reflected by total internal reflection at the tapered sidewall of the tapered TIR optic into a light distribution having peak intensity at an angle of at least 55 degrees respective to the symmetry axis.

17. The beam spreading optical element of claim 13 wherein the tapered sidewall of the tapered TIR optic has grooves and/or ridges with each groove or ridge extending between the apex and the perimeter of the base.

18. The beam spreading optical element of claim 13 wherein the optical condensers comprise the same material as the tapered TIR optic and the beam spreading optical element is formed as a single element.

19-20. (canceled)

21. A luminaire comprising:
a beam spreading optical element as set forth in claim 13; and
light emitting diodes (LEDs) coupled with the input apertures of the optical condensers of the beam spreading optical element.

22. The luminaire of claim 21 wherein the LEDs are configured to emit ultraviolet light and the design-basis wavelength is in the range 200-400 nm.

23. The luminaire of claim 21 further comprising:
peripheral white LEDs configured to emit white light, the peripheral white LEDs being disposed around the base of the beam spreading optical element, wherein the peripheral white LEDs are not optically coupled with the beam spreading optical element.

24-25. (canceled)

26. The luminaire of claim 21 further comprising:
an annular reflector surrounding the beam spreading optical element.

27. A method of manufacturing a beam spreading optical element, the method comprising:
molding the beam spreading optical element of a material having a refractive index at a design-basis wavelength, the molding forming the beam spreading optical element as a single molded piece including a tapered total internal reflection (TIR) optic and N optical condensers connected with the tapered TIR optic where N is at least three, wherein:
the tapered TIR optic has a base and an apex and a tapered sidewall that extends from a perimeter of the base to the apex; and
each optical condenser is connected to the base of the tapered TIR element and is configured to condense light of the design-basis wavelength received at an input aperture of the optical condenser into a condensed light beam that passes into the tapered TIR optic and intersects the tapered sidewall of the tapered TIR optic at an angle effective for light beam to be reflected by total internal reflection at the tapered sidewall of the tapered TIR optic.
